(19) United States
(12) Patent Application Publication
(10) Pub. No.: US 2010/0144818 A1
(43) Pub. Date: Jun. 10, 2010

(54) 1-BENZYLPYRAZOLE DERIVATIVES, PREPARATION THEREOF AND THERAPEUTIC USE THEREOF

(75) Inventors: Francis BARTH, Saint Georges D'Orques (FR); Laurent BOULU, Juvignac (FR); Joseph MILLAN, Juvignac (FR); Murielle RINALDI-CARMONA, Saint Georges D'Orques (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/630,470

(22) Filed: Dec. 3, 2009

Related U.S. Application Data
(63) Continuation of application No. PCT/FR2008/000739, filed on Jun. 2, 2008.

(30) Foreign Application Priority Data
Jun. 4, 2007 (FR) 0703972

(51) Int. Cl. A61K 31/415; C07D 231/12; A61P 41/00

(52) U.S. Cl. 514/406; 548/377.1

(57) ABSTRACT

The present invention relates to compounds corresponding to formula (I):

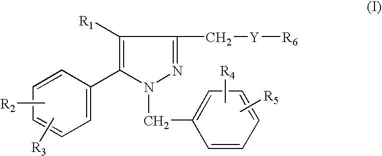

in which Y, R₁, R₂, R₃, R₄, R₅ and R₆ are as defined herein. The present invention also relates to the methods of preparation and the therapeutic applications of the compounds of formula (I).

This application is a continuation of International application No. PCT/FR2008/000,739, filed Jun. 2, 2008, which is incorporated herein by reference in its entirety; which claims the benefit of priority of French patent application Ser. No. 07/03,972, filed Jun. 4, 2007.

The present invention relates to 1-benzylpyrazole derivatives, the preparation thereof and the therapeutic use thereof.

1-Benzylpyrazole derivatives are known in the literature, notably European patent EP-B-868 420 and its American equivalent U.S. Pat. No. 5,925,768 describe compounds of formula:

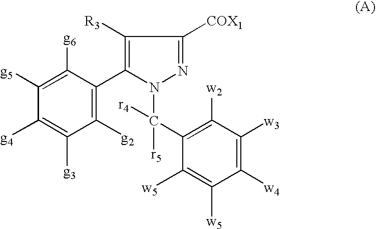

These compounds have affinity for the human CB₂ receptors.

Patent application FR 2 887 550 describes compounds of formula:

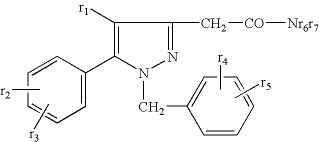

which are ligands of the CB₂ cannabinoid receptors.

Compounds have now been found that are antagonists of the CB₂ cannabinoid receptors and easily cross the intestinal barrier.

The present invention relates to compounds corresponding to formula (I):

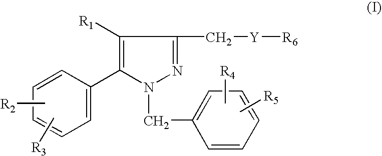

in which:
Y represents a group selected from:
   i) —N(R₇)CO—,
   ii) —N(R₇)CO—N(R₇)—,
   iii) —OCO—,
   iv) —N(R₇)S(O)ₙ—;
R₁ represents a hydrogen atom or a (C₁-C₄)alkyl group;
R₂ and R₄ represent, each independently of one another, a hydrogen or halogen atom, a (C₁-C₄)alkyl, (C₁-C₄)alkoxy or trifluoromethyl group;
R₃ and R₅ represent, each independently of one another, a halogen atom or a (C₁-C₄)alkyl, (C₁-C₄)alkoxy, trifluoromethyl, trifluoromethoxy, cyano or S(O)ₘAlk group;
R₆ represents a group selected from:
   a (C₁-C₆)alkyl group, unsubstituted or substituted one or more times with one or more substituents selected independently from a halogen atom or a hydroxy, (C₁-C₄)alkoxy or trifluoromethoxy group;
   a phenyl, unsubstituted or substituted one or more times with R₈;
   a benzyl or benzhydryl;
   a heterocyclic radical selected from: thienyl, furyl or pyrrolyl, said radicals being unsubstituted or substituted with a halogen atom, a (C₁-C₄)alkyl or trifluoromethyl group;
   a C₃-C₁₂ nonaromatic carbocyclic radical, unsubstituted or substituted one or more times with a halogen atom or a (C₁-C₄)alkyl, (C₁-C₄)alkoxy, hydroxy or cyano group;
   a (C₃-C₇)cycloalkylmethyl group, unsubstituted or substituted on the cycloalkyl with one or more (C₁-C₄)alkyl groups;
   an aryloxymethyl group, unsubstituted or substituted on the methyl with one or two alkyl groups, in which the term aryloxy represents a phenoxy group, unsubstituted or substituted one or more times with R₈;
R₇ represents a hydrogen atom or a (C₁-C₄)alkyl group;
R₈ represents a halogen atom; a (C₁-C₄)alkyl; trifluoromethyl; cyano; (C₁-C₄)alkoxy; trifluoromethoxy; phenyl; (C₃-C₇)cycloalkyl or NHS(O)ₙAlk group;
n represents 1 or 2;
m represents 0, 1 or 2;
Alk represents a (C₁-C₄)alkyl.

The compounds of formula (I) can be in the form of bases or of salts of addition to acids. Said salts of addition form part of the invention.

These salts are advantageously prepared with pharmaceutically acceptable acids, but the salts of other acids that can be used for example for the purification or isolation of the compounds of formula (I) also form part of the invention.

The compounds of formula (I) can also be in the form of hydrates or solvates, namely in the form of associations or of combinations with one or more molecules of water or with solvent. Said hydrates and solvates also form part of the invention.

Moreover, the compounds of formula (I) according to the invention, just as they are or in radiolabeled form, can be used as pharmacological tools in humans or animals for the detection and labeling of CB₂ cannabinoid receptors. We may mention in particular compounds (I) bearing one or more labeled atoms selected from ³H, ¹¹C, ¹⁴C, ¹⁸F, ³⁵S, ⁷⁶Br, ¹²³I, ¹²⁵I, ¹³¹I.

The compounds of formula (I) can contain one or more asymmetric carbon atoms. They can therefore exist in the form of enantiomers or of diastereoisomers. These enantiomers, diastereoisomers, and mixtures thereof, including racemic mixtures, form part of the invention.

Within the scope of the present invention, the following terms are used:
a halogen atom: a fluorine, a chlorine, a bromine or an iodine, with chlorine and fluorine being preferred;
a (C₁-C₄)alkyl or (C₁-C₆)alkyl group: a saturated, linear or branched C₁-C₄ or C₁-C₆ aliphatic group respectively. As examples, we may mention the methyl, ethyl, propyl, isopropyl, butyl, isobutyl, sec-butyl, tert-butyl groups, as well as the pentyl, isopentyl, hexyl, isohexyl groups, for the (C₁-C₆) alkyl groups;
a (C₁-C₄)alkoxy group: an O-alkyl radical, where the alkyl group is as described previously;
a C₃-C₁₂ nonaromatic carbocyclic radical: a monocyclic radical or a di- or tricyclic radical, condensed or bridged; monocyclic radical means a cycloalkyl, i.e. a cyclic alkyl group, for example cyclopropyl, cyclobutyl, cyclopentyl, cyclohexyl, cycloheptyl, the cyclopentyl, cyclohexyl and cycloheptyl radicals being preferred; di- or tricyclic radical, condensed or bridged, means for example bicyclo[2.2.1]heptyl, bicyclo[2.2.2]octanyl, bicyclo[3.2.1]octanyl, adamantyl.

Among the compounds of formula (I) according to the invention, the following are distinguished:

compounds of formula (IA) in which Y represents a group —N(R₇)CO—;

compounds of formula (IB) in which Y represents a group —N(R₇)CON(R₇)—;

compounds of formula (IC) in which Y represents a group —OCO—;

compounds of formula (ID) in which Y represents a group —N(R₇)S(O)ₙ—.

Among the compounds of formula (I) according to the invention, we may mention the preferred compounds that are defined as follows:
R₁ represents hydrogen;
R₂ and R₃ represent a chlorine atom at 3 or at 4, a trifluoromethyl group at 4, or two chlorine atoms at 3 and 4;
R₄ and R₅ represent a chlorine atom at 2, 3 or 4, a trifluoromethyl group at 4, or a chlorine atom at 3 and a methyl group at 4.

Among the 4 families of compounds, those of formula (IA) and of formula (ID) are preferred.

Among the compounds of formulas (IA) and (ID), the compounds are preferred whose substituents have the following definitions:
R₁ represents hydrogen;
R₂ and R₃ represent a chlorine atom at 3 or at 4, a trifluoromethyl group at 4, or two chlorine atoms at 3 and 4;
R₄ and R₅ represent a chlorine atom at 2, 3 or 4, a trifluoromethyl group at 4, or a chlorine atom at 3 and a methyl group at 4.
R₆ represents a group selected from:
   a (C₁-C₆)alkyl group, unsubstituted or substituted one or more times with one or more substituents selected independently from a halogen atom or a hydroxy, (C₁-C₄)alkoxy or trifluoromethoxy group;
   a C₃-C₁₂ nonaromatic carbocyclic radical, unsubstituted or substituted one or more times with a halogen atom or a (C₁-C₄)alkyl, (C₁-C₄)alkoxy, hydroxy or cyano group.

Quite particularly, the following compounds are preferred:
N-((1-(3,4-dichlorobenzyl)-5-(3,4-dichlorophenyl)-1H-pyrazol-3-yl)methyl)-2,2-dimethylpropanamide;
and N-((1-(3,4-dichlorobenzyl)-5-(3,4-dichlorophenyl)-1H-pyrazol-3-yl)methyl)-2-methylpropane-2-sulfonamide.

According to the invention, the compounds of formula (I) can be prepared by the following method. This method is characterized in that:

a compound of formula:

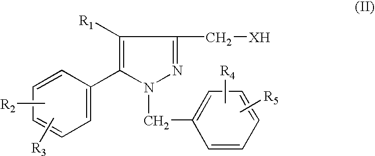

in which X represents an oxygen atom, an NH or N(R₇) group and R₁, R₂, R₃, R₄, R₅ are as defined above for the compounds of formula (I), is treated:
a) either with a functional derivative of an acid of formula R₆COOH (III) in which R₆ is as defined above for (I) to obtain a compound of formula (IA) or (IC) in which Y represents the value i) or ii);
b) or with an isocyanate of formula R₆N═C═O (IV) in which R₆ is as defined above for (I), to obtain a compound of formula (IB) in which Y represents the value ii);
c) or with a halogenated derivative of formula R₆S(O)ₙHal in which Hal represents a halogen atom, preferably chlorine, and R₆ is as defined above for (I), to obtain a compound of formula (ID) in which Y has the value iv).

Starting from a compound of formula (ID) in which Y=—N(R₇)SO—, a compound of formula (ID) can be prepared in which Y=—N(R₇)SO₂—, by oxidation, for example by the action of metachloroperbenzoic acid.

Functional derivative of an acid of formula R₆COOH means an acid chloride, an anhydride or even the free acid suitably activated for example with benzotriazol-1-yloxytris(dimethylamino)phosphonium hexafluorophosphate (BOP) or dicyclohexylcarbodiimide (DCC).

The compounds of formula (I) in which Y represents a group —N(R₇)CO— or —N(R₇)CON(R₇)— can be prepared from the corresponding compounds of formula (I) in which Y represents a group —NHCO— or —NHCONH— by a method selected from the methods known by a person skilled in the art. Among the latter, we may mention alkylation by a methyl halide or the formation of a carbamate by the action of ethyl chloroformate followed by reduction by LiAlH₄.

According to a variant of the method of the invention, the compounds of formula (I_{B}) can be obtained by a method characterized in that:

c₁) a compound of formula (II) is treated with an ester of formula HalCOOAr (V) in which Hal represents a halogen atom and Ar represents an aryl group such as phenyl or 4-nitrophenyl;

c₂) the carbamate thus obtained of formula:

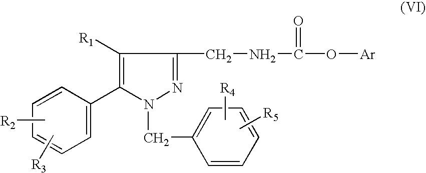

is treated with an amine of formula: R₆R₇NH (VII) in which R₆ and R₇ are as defined for a compound of formula (IB).

Stage a) is carried out in an aprotic solvent such as dichloromethane, THF or DMF at a temperature between 0° C. and the boiling point of the solvent.

Stage b) is carried out in a solvent such as dichloromethane at room temperature.

Stage c₁) is carried out in an aprotic solvent such as dichloromethane at room temperature.

Stage c₂) is carried out in an aprotic solvent such as dichloromethane, at a temperature between room temperature and the boiling point of the solvent.

The compounds of formula (II) are prepared according to the following reaction scheme:

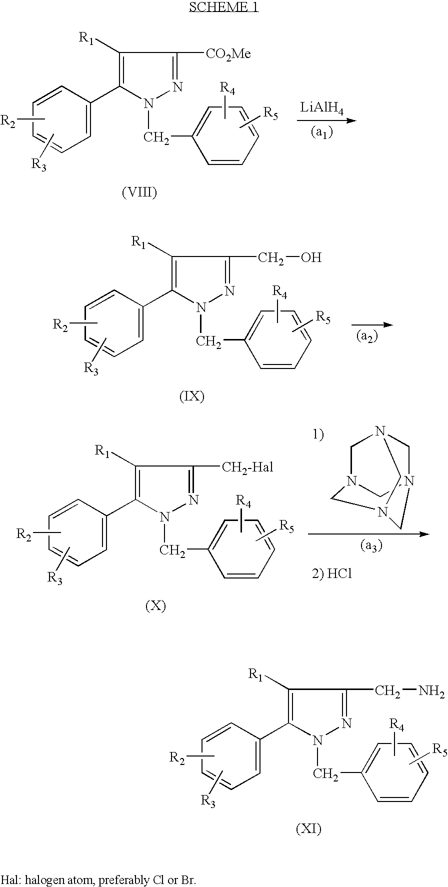

When X represents an NH group, the compound of formula (II) is represented by the compound of formula (XI).

When X represents an oxygen atom, the compound of formula (II) is represented by the compound of formula (IX).

In the first stage (a₁), the ester of formula (VIII) is reduced by the action of a reducing agent such as LiAlH₄.

In the next stage (a₂), the hydroxymethylated derivative (IX) obtained is treated with an agent such as PCl₅, PBr₃, HBr or BBr₃ to form the halomethylated derivative of formula (X).

Finally, the compound of formula (XI) is obtained in the course of stage (a₃) by treating the compound of formula (X) successively with 1,3,5,7-tetraazatricyclo[3.3.1^{3,7}]decane (or hexamethylene tetramine) then with a strong acid such as hydrochloric acid.

The compounds of formula (VIII) are obtained according to the method of preparation described in patent EP-B-868 420.

The compounds of formula (XI) are novel.

Thus the invention, according to another of its aspects, also relates to the compounds of formula (XII):

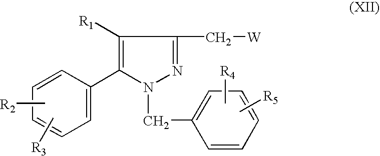

in which:

W represents a hydroxyl or amino group;

R₁ represents a hydrogen atom or a (C₁-C₄)alkyl group;

R₂ and R₄ represent, each independently of one another, a hydrogen or halogen atom, or a (C₁-C₄)alkyl, (C₁-C₄)alkoxy or trifluoromethyl group;

R₃ and R₅ represent, each independently of one another, a halogen atom or a (C₁-C₄)alkyl, (C₁-C₄)alkoxy or trifluoromethyl group.

The compounds of formula (XII) can be in the form of bases or of salts of addition to acids. Said salts of addition form part of the invention.

The compounds of formula (XII) can also be in the form of hydrates or solvates, namely in the form of associations or of combinations with one or more molecules of water or with solvent. Said hydrates and solvates also form part of the invention. These compounds can be used as intermediates for synthesis of the compounds of formula (I) according to the invention.

Preferably, the compounds of formula (XII) correspond to those whose substituents have the following definitions:
R₁ represents a hydrogen atom;
R₂ and R₃ represent a chlorine atom at 3 or at 4, a trifluoromethyl group at 4, or two chlorine atoms at 3 and 4;
R₄ and R₅ represent a chlorine atom at 2, 3 or 4, a trifluoromethyl group at 4, or a chlorine atom at 3 and a methyl group at 4.
R₆ represents a group selected from:
   a (C₁-C₆)alkyl group, unsubstituted or substituted one or more times with one or more substituents selected independently from a halogen atom or a hydroxy, (C₁-C₄)alkoxy or trifluoromethoxy group;
   a C₃-C₁₂ nonaromatic carbocyclic radical, unsubstituted or substituted one or more times with a halogen atom or a (C₁-C₄)alkyl, (C₁-C₄)alkoxy, hydroxy or cyano group.

The following examples describe the preparation of certain compounds according to the invention. These examples are not exhaustive and are only intended to illustrate the present invention. The numbers of the compounds in the examples refer to those given in the table hereunder, which illustrates the chemical structures and the physical properties of some compounds according to the invention.

The following abbreviations are used in the examples:

RT: room temperature

(dec.): decomposition

TEA: triethylamine

THF: tetrahydrofuran

DMF: dimethylformamide

DCM: dichloromethane

DMSO: dimethylsulfoxide

BOP: benzotriazol-1-yloxytris(dimethylamino)phosphonium/hexafluorophosphate

K₂SO₄/KHSO₄: buffer pH 2 (KHSO₄ 0.12 M and K₂SO₄ 0.185 M)

EtOAc: ethyl acetate

PyBOP: benzotriazol-1-yloxytris(pyrrolidino) phosphonium hexafluorophosphate

LiHMDS: lithium salt of hexamethyldisilazane

MeOH: methanol

EtOH: ethanol

Et₂O: ethyl ether

AcOH: acetic acid

m.p.: melting point.

For the nuclear magnetic resonance (NMR) spectra recorded at 200 MHz in DMSO-d₆, the signals observed are expressed thus: d: doublet; bd: broad doublet; dd: doublet of doublets; s: singlet; bs: broad singlet; t: triplet.

The compounds according to the invention are analyzed by coupling LC/UV/MS (liquid chromatography/UV detection/mass spectrometry). The molecular peak (MN⁺) and the retention time (t) in minutes are measured.

Conditions (A):

Xterra Waters® MS C18 column, marketed by Waters, 2.1×30 mm, 3.5 μm, at room temperature, flow 1 mL/min.

The eluent has the following composition:

solvent A: 0.025% of trifluoroacetic acid (TFA) in water;

solvent B: 0.025% of TFA in acetonitrile.

Gradient: The percentage of solvent B varies from 0 to 100% in 2 minutes with a plateau at 100% of B for 1 minute.

UV detection is effected between 210 nm and 400 nm and mass detection in chemical ionization mode is effected at atmospheric pressure.

Conditions (B):

Symmetry C18 column, 2.1×50 mm, 3.5 μm, at 30° C., flow 0.4 mL/min.

The eluent has the following composition:

solvent A: 0.005% of trifluoroacetic acid (TFA) in water at pH 3.15;

solvent B: 0.005% of TFA in acetonitrile.

Gradient:

| Time (min) | % A | % B |
|---|---|---|
| 0 | 100 | 0 |
| 10 | 10 | 90 |
| 15 | 10 | 90 |
| 16 | 100 | 0 |
| 20 | 100 | 0 |

Column temperature: 30° C., flow 0.4 ml/min.

UV detection is effected at λ=210 nM and mass detection in chemical ionization mode (ESI: ElectroSpray Ionization) is positive.

Conditions (C):

Symmetry C18 column, 2.1×50 mm, 3.5 μm, at 30° C., flow 0.4 mL/min.

The eluent has the following composition:

solvent A: 0.005% of trifluoroacetic acid (TFA) in water at pH 3.1;

solvent B: 0.005% of TFA in acetonitrile.

Gradient:

| Time (min) | % A | % B |
|---|---|---|
| 0 | 100 | 0 |
| 10 | 10 | 90 |
| 15 | 10 | 90 |
| 16 | 100 | 0 |
| 20 | 100 | 0 |

Column temperature: 30° C., flow 0.4 ml/min.

UV detection is effected at λ=210-220 nM and mass detection in chemical ionization mode (ElectroSpray Ionization, ESI) is positive.

Conditions (D):

XTerra MS C18 column, 2.1×50 mm, 3.5 μm, at 30° C., flow 0.4 mL/min.

The eluent has the following composition:

solvent A: 10 mM of ammonium acetate in water at pH 7;

solvent B: acetonitrile.

Gradient:

| Time (min) | % A | % B |
|---|---|---|
| 0 | 100 | 0 |
| 10 | 10 | 90 |
| 15 | 10 | 90 |
| 16 | 100 | 0 |
| 20 | 100 | 0 |

Column temperature: 30° C., flow 0.4 ml/min.

UV detection is effected at λ=210 nM and mass detection in chemical ionization mode (ESI: ElectroSpray Ionization) is positive.

### EXAMPLE 1

### Compound 19

### N-((1-(3-Chloro-4-methylbenzyl)-5-(3,4-dichlorophenyl)-1H-pyrazol-3-yl)-methyl)-1-methylcyclohexanecarboxamide

### A) (1-(3-Chloro-4-methylbenzyl)-5-(3,4-dichlorophenyl)-1H-pyrazol-3-yl)methanol

Dissolve 4 g of 1-(3-chloro-4-methylbenzyl)-5-(3,4-dichlorophenyl)-1H-pyrazole-3-methyl carboxylate in 100 ml of THF and cool between 0° C. and −5° C., then add 0.6 g of LiAlH₄ a little at a time, maintaining the same low temperature. Leave to return to RT and stir for 30 minutes. Carry out hydrolysis at 0° C. with NaOH N (100 ml). Filter, wash the filtrate with THF and then evaporate. The oil obtained is taken up in ether, it is washed with water, with NaCl solution, then it is dried over Na₂SO₄ and evaporated to dryness to obtain 3.5 g of the expected compound.

NMR (200 MHz) in DMSO-d₆: 7.7 ppm: d: 1H, 7.6 ppm: d: 1H, 7.4 ppm: dd: 1H, 7.3 ppm: d: 1H; 7 ppm: d: 1H, 6.8 ppm: dd: 1H, 6.4 ppm: s: 1H, 5.3 ppm: s: 2H, 5.1 ppm: t: 1H, 4.4 ppm: d: 2H, 2.2 ppm: s: 3H.

### B) 3-(Chloromethyl)-1-(3-chloro-4-methylbenzyl)-5-(3,4-dichlorophenyl)-1H-pyrazole

Dissolve 3.45 g of compound of the compound from the preceding stage in 100 ml of DCM. Cool on an ice bath and add, a little at a time, at a temperature below 10° C., 1.96 g of PCl₅. Stir for 3 hours at RT, then pour into a mixture of water and ice and continue stirring for 3 hours. Leave to settle, then wash the organic phase with a solution of NaHCO₃ and then water. Dry over Na₂SO₄, then evaporate to dryness. The oil obtained is purified by silica chromatography, eluting with EtOAc/cyclohexane mixture (25/75; v/v). We obtain 2.35 g of the expected compound in the form of oil, which crystallizes. M.p.=88° C.

### C) (1-(3-Chloro-4-methylbenzyl)-5-(3,4-dichlorophenyl)-1H-pyrazol-3-yl)methanamine hydrochloride

Dissolve 2.3 g of the compound from the preceding stage in 200 ml of CHCl₃ and add 0.8 g of 1,3,5,7-tetraazatricyclo[3.3.1^{3,7}]decane. After stirring for 5 days at RT, add 150 ml of ether, drain the precipitate that forms and then dissolve it in 50 ml of ethanol. Add 2 ml of concentrated HCl, then heat at 50° C. for 3 hours. Concentrate the mixture to half the volume, then add 50 ml of ether. Drain, wash with ether and dry to obtain 1.78 g of the expected compound in the form of a base.

To prepare the hydrochloride, dissolve in 50 ml of ethanol, add 2 ml of concentrated HCl, then heat at 50° C. for 3 hours. Concentrate the mixture under vacuum, drain, wash with ether and then dry; we obtain 1.88 g of the expected hydrochloride.

NMR (200 MHz) in DMSO-d₆: 8.6 ppm: bs: 1H, 7.7 ppm: d: 1H, 7.6 ppm: d: 1H, 7.4 ppm: dd: 1H, 7.3 ppm: d: 1H, 7.1 ppm: d: 1H, 6.9 ppm: dd: 1H, 6.7 ppm: s: 1H; 6 ppm: bs: 2H, 5.4 ppm: s: 2H; 4 ppm: 2 d: 2H, 2.3 ppm: s: 3H.

### D) N-((1-(3-Chloro-4-methylbenzyl)-5-(3,4-dichlorophenyl)-1H-pyrazol-3-yl)methyl)-1-methylcyclohexanecarboxamide

Mix 417 mg of the compound from the preceding stage, 142 mg of 1-methylcyclohexanecarboxylic acid and 580 mg of BOP in 50 ml of DMF and then add triethylamine to reach pH 10.

Stir for 20 hours at room temperature.

The product obtained is purified by silica chromatography, eluting with DCM/MeOH mixture (97.5/2.5; v/v). We obtain 250 mg of the expected compound, m.p.=63° C.

### EXAMPLE 2

### Compound 60

### N-(tert-Butyl)-N′-((1-(3-chloro-4-methylbenzyl)-5-(3,4-dichlorophenyl)-1H-pyrazol-3-yl)methyl)urea

Dissolve 0.5 g of the compound obtained in stage C of Example 1 in 30 ml of DCM and add 0.137 ml of 2-isocyanato-2-methylpropane and then a sufficient amount of triethylamine to reach pH 9. Stir at RT overnight. After adding 10 ml of water, decant. Wash the organic phase successively with aqueous solutions of NaHCO₃, K₂SO₄/KHSO₄, then water. Dry over Na₂SO₄, then evaporate to dryness. The residue is taken up in EtOAc/cyclohexane mixture (50/50; v/v). The expected compound is precipitated in the form of a white solid: m=360 mg, m.p.=181° C.

### EXAMPLE 3

### Compound 46

### N-((1-(3-Chlorobenzyl)-5-(3,4-dichlorophenyl)-1H-pyrazol-3-yl)methyl)-2,2-dimethylpropanamide

### A) Sodium 1-(3,4-dichlorophenyl)-4-methoxy-3,4-dioxobut-1-en-1-olate

Prepare a solution of sodium methylate by slowly adding 23 g of sodium to 1 liter of methanol, cool on an ice bath and add 188.97 g of 1-(3,4-dichlorophenyl)ethanone and then 149.14 g of ethyl oxalate in 250 ml of methanol. A precipitate forms. Add 500 ml of methanol, then stir for 2 hours. Add 500 ml of Et₂O and continue stirring for 30 minutes. Filter the precipitate that has formed, then dry it, to obtain 297 g of the expected compound.

### B) 5-(3,4-Dichlorophenyl)-1H-pyrazole-3-methyl carboxylate

Put 123.43 g of the compound obtained in the preceding stage in 800 ml of AcOH, add dropwise 25.60 ml of hydrazine hydrate and heat overnight with reflux. Pour the reaction mixture into iced water. Drain the precipitate that has formed, then wash it with water. It is then taken up in Et₂O, the organic phase is washed with saturated NaCl solution and then dried over Na₂SO₄, evaporated and filtered. We obtain 91.85 g of the expected compound.

### C) 1-(3-Chlorophenyl)-5-(3,4-dichlorophenyl)-1H-pyrazole-3-methyl carboxylate

Put 14.50 g of the compound obtained in the preceding stage in 250 ml of toluene and add 2.35 g of NaH a pinch at a time, then heat at 65° C. for 1 hour. Cool to RT and slowly add 7.72 ml of 3-chlorobenzyl bromide. Heat the reaction mixture under reflux for 20 hours, then neutralize it with a saturated solution of NH₄Cl. Leave the organic phase to settle, then wash it with saturated NaCl solution. Dry over Na₂SO₄ and then evaporate. Dissolve the product obtained in EtOAc, then wash with NaHCO₃ saturated solution, K₂SO₄/KHSO₄ solution, and then water. Dry over Na₂SO₄ and evaporate, to obtain 64.37 g of the expected compound.

### D) 1-(1-(3-Chlorobenzyl)-5-(3,4-dichlorophenyl)-1H-pyrazol-3-yl)methanol

Dissolve 24.26 g of the compound obtained in the preceding stage in 500 ml of THF under nitrogen atmosphere and cool to −5° C. Add 1.47 g of the LiAlH₄ in small fractions at a temperature below 0° C. Leave to return to RT and then stir for 1 hour. Carry out hydrolysis of the reaction mixture at 0° C. with 1N NaOH. Filter, then wash the insoluble matter with THF. The filtrates are combined, evaporated to dryness, and then taken up in ether. The organic phase is washed with a saturated solution of NaCl and it is then dried over Na₂SO₄ and evaporated to dryness. The product obtained is purified by silica chromatography, eluting with EtOAc/cyclohexane mixture (50/50; v/v). We obtain 6.82 g of the expected compound, m.p.=95° C.

### E) 1-(3-Chlorobenzyl)-3-(chloromethyl)-5-(3,4-dichlorophenyl)-1H-pyrazole

Dissolve 6.7 g of the compound obtained in the preceding stage in 200 ml of DCM and cool to between 0° C. and −10° C. Add 3.98 g of PCl₅ a pinch at a time at −10° C. then stir for 4 hours at RT. Pour the reaction mixture onto ice and leave to stand overnight. The organic phase is decanted and then extracted again with 50 ml of DCM. The extracts are combined, dried over Na₂SO₄ and evaporated to dryness. We obtain 7.41 g of the expected compound.

### F) 1-(1-(3-Chlorophenyl)-5-(3,4-dichlorophenyl)-1H-pyrazol-3-yl)methanamine hydrochloride

Dissolve 7.3 g of the compound obtained in the preceding stage in 500 ml of chloroform and add 2.65 g of 1,3,5,7-tetraazatricyclo[3.3.1^{3,7}]decane. After stirring for 5 days, add 500 ml of ether. After 2 hours, drain the precipitate that has formed. Dissolve the product obtained in 50 ml of EtOH, then add 2 ml of concentrated HCl and heat at 60° C. for 3 hours. After evaporating to dryness, it is taken up again in ethyl chloride. The precipitate formed is drained and then dried, to give 4.67 g of the expected compound. M.p.=150° C.

### G) N-((1-(3-Chlorobenzyl)-5-(3,4-dichlorophenyl)-1H-pyrazol-3-yl)methyl)-2,2-dimethylpropanamide

Dissolve 0.6 g of the compound obtained in the preceding stage and 0.18 g of pivalic acid in 50 ml of DMF, then add 0.92 g of PyBOP and 0.45 ml of triethylamine and stir overnight at RT. After evaporating the DMF, the residue is taken up in EtOAc. It is washed with a saturated solution of NaHCO₃, K₂SO₄/KHSO₄ solution and then water; it is dried over Na₂SO₄, then evaporated to dryness. The product obtained is purified by silica chromatography, eluting with DCM/MeOH mixture (95/5; v/v). We obtain 0.31 g of the expected compound.

NMR: 1.12 ppm: s: 9H, 4.25 ppm: d: 2H, 5.34 ppm: s: 2H, 6.33 ppm: s: 1H, 6.85-7.45 ppm: m: 5H, 7.61 ppm: d: 1H, 7.71 ppm: d: 1H, 7.98 ppm: t: 1H.

### EXAMPLE 4

### Compound No. 55

### (1-(3,4-Dichlorobenzyl)-5-(4-trifluoromethylphenyl)-1H-pyrazol-3-yl) methyl pivalate

### A) Lithium 1-(4-(trifluoromethylphenyl)-4-ethoxy-3,4-dioxobut-1-en-1-olate

Under a nitrogen atmosphere, dissolve 24.45 g of LiHMDS in 100 ml of anhydrous THF and cool to −60° C. In addition, dissolve 25 g of 1-(4-trifluoromethylphenyl)ethanone in 50 ml of Et₂O. Add the solution of ketone dropwise to the solution of LiHMDS at −60° C., then allow the temperature to rise to −30° C. and quickly add 19.85 ml of diethyl oxalate. After one night at RT, the reaction mixture is evaporated to dryness and then taken up in ether. The precipitate that forms is drained, washed with ether and then dried. We obtain 33.28 g of the expected compound.

### B) 5-(4-Trifluoromethylphenyl)-1H-pyrazol-3-ethyl carboxylate

Dissolve 33.2 g of the compound obtained in the preceding stage in 400 ml of AcOH and add dropwise 6.33 ml of hydrazine hydrate, then heat under reflux for 5 hours, with stirring. After 1 night at RT, pour the mixture into iced water and drain the precipitate that forms after 1 hour. Wash with water and dry, then take up in DCM. Dry over Na₂SO₄ and evaporate to dryness. We obtain 30.37 g of the expected compound.

### C) 1-(3,4-Dichlorobenzyl)-5-(4-trifluoromethylphenyl)-1H-pyrazole-3-ethyl carboxylate

Starting with 30.3 g of the compound obtained in the preceding stage, this compound is prepared according to the method described in Example 3, stage C. We obtain 36.24 g of the expected compound.

### D) 1-(1-(3,4-Dichlorobenzyl)-5-(4-trifluoromethylphenyl)-1H-pyrazol-3-yl) methanol

This compound is prepared by following the method described in Example 3, stage D. Starting with 33 g of the compound from the preceding stage, we obtain 23.10 g of the expected compound, m.p.=96° C.

### E) (1-(3,4-Dichlorobenzyl)-5-(4-trifluoromethylphenyl)-1H-pyrazol-3-yl) methyl pivalate

Dissolve 1 g of the compound obtained in the preceding stage in 50 ml of DMF. Add 0.37 ml of pivaloyl chloride, then 0.52 ml of triethylamine and stir at RT for 20 hours. Evaporate the solvent under vacuum and then take up in 50 ml of EtOAc. Wash with saturated solution of NaHCO₃, then with K₂SO₄/KHSO₄ and then with saturated NaCl solution. Dry over Na₂SO₄, then evaporate to dryness. The product obtained is purified by silica chromatography, eluting with EtOAc/cyclohexane mixture (25/75; v/v). We obtain 0.89 g of the expected compound in the form of oil, which crystallizes, m.p.=92° C.

The intermediates described in Table 1 below are prepared following the procedure in stage C) of Example 1. When the compound is characterized by LC/UV/MS, operating conditions (A), (B), (C) or (D) are specified.

TABLE 1

| | |
|---|---|
| | (II) |
| 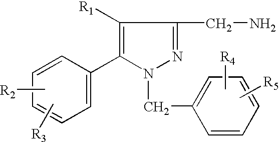 | |

| | | | Characterization | |
|---|---|---|---|---|
| | | | m.p. ° C. or | |
| R₁ | R₂, R₃ | R₄, R₅ | LC/MS | Salt |
| H | 4-Cl | 2,4-diCl | 176° C. | HCl |
| H | 3,4-diCl | 4-Cl | 237° C. | HCl |
| H | 4-Cl | 3,4-diCl | 214° C. | HCl |
| Me | 4-Cl | 2,4-diCl | 172° C. | HCl |
| H | 3-Cl | 3,4-diCl | 63° C. | HCl |
| H | 3,4-diCl | 4-CF₃ | 382° C. | HCl |
| H | 3-Cl | 3-Cl | 165° C. | HCl |
| H | 4-Cl | 4-Cl | 229° C. | HCl |
| H | 3,4-diCl | 3,4-diCl | 205° C. | HCl |
| H | 3,4-diCl | 3-Cl, 4-OMe | MH⁺ = 396 | HCl |
| | | | t = 7.25 min | |
| | | | (C) | |
| H | 3,4-diCl | 3-Cl, 4-OCF₃ | MH⁺ = 450 | HCl |
| | | | t = 7.25 min | |
| | | | (C) | |
| H | 3,4-diCl | 3-Cl, 4-CF₃ | MH⁺ = 434 | HCl |
| | | | t = 7.47 min | |
| | | | (C) | |
| H | 3,4-diCl | 3-CF₃, 4-Cl | MH⁺ = 434 | HCl |
| | | | t = 7.46 min | |
| | | | (C) | |
| H | 4-OMe | 3,4-diCl | MH⁺ = 362 | HCl |
| | | | t = 6.62 min | |
| | | | (C) | |

### EXAMPLE 5

### Compound No. 60

### N-((1-(3,4-Dichlorobenzyl)-5-(3,4-dichlorophenyl)-1H-pyrazol-3-yl)methyl)-2,2-dimethylpropanamide

Put 0.50 g of 1-(1-(3,4-dichlorobenzyl)-5-(3,4-dichlorophenyl)-1H-pyrazol-3-yl)methanamine, described in table I, and 0.15 g of pivaloic acid in 50 ml of DMF in the presence of 0.68 g of PyBOP and add 0.28 ml of TEA. After stirring overnight at room temperature, evaporate the solvent and then take up in EtOAc, wash with a saturated solution of NaHCO₃, then with K₂SO₄/KHSO₄. Dry over Na₂SO₄, then evaporate to dryness. Purify by silica chromatography, eluting with EtOAc/cyclohexane mixture (50/50; v/v). We obtain 0.36 g of the expected compound. M.p.=56.5° C.

NMR: 1.09 ppm: s: 9H, 4.23 ppm: d: 2H, 5.32 ppm: bs: 2H, 6.31 ppm: s: 1H, 6.96 ppm: dd: 1H, 7.27 ppm: d: 1H, 7.61 ppm: d: 1H, 7.69 ppm: d: 1H; 7.95 ppm: t: 1H.

### EXAMPLE 6

### Compound No. 89

### N-((1-(3,4-Dichlorobenzyl)-5-(3,4-dichlorophenyl)-1H-pyrazol-3-yl)methyl)-2-methylpropane-2-sulfinamide

Put 1.2 g of 1-(1-(3,4-dichlorobenzyl)-5-(3,4-dichlorophenyl)-1H-pyrazol-3-yl)methanamine described in table I, and 0.44 ml of 2-methylpropane-2-sulfinyl chloride in 50 ml of DMF and add 1.24 ml of TEA. Stir for 16 hours. Evaporate the solvent and then purify by silica chromatography, eluting with cyclohexane and then with a cyclohexane/EtOAc gradient from 99/1 to 70/30. We obtain 0.85 g of the expected compound.

NMR: 1.12 ppm: s: 9H, 4.15 ppm: m: 2H, 5.37 ppm: s: 2H, 5.71 ppm: t: 1H, 6.52 ppm: s: 1H, 6.99 ppm: dd: 1H, 7.27 ppm: d: 1H, 7.36 ppm: dd: 1H, 7.57 ppm: d: 1H, 7.63 ppm: d: 1H, 7.72 ppm: d: 1H.

### EXAMPLE 7

### Compound No. 90

### N-((1-(3,4-Dichlorobenzyl)-5-(3,4-dichlorophenyl)-1H-pyrazol-3-yl)methyl)-2-methylpropane-2-sulfonamide

Dissolve 0.68 g of metachloroperbenzoic acid in 55 ml of DCM, cool to between 0° C. and −5° C. and then add 0.55 g of the compound obtained in the preceding example in 5 ml of DCM. Allow to return to room temperature and then stir overnight. Pour the reaction mixture into 50 ml of 1N NaOH, then decant. The organic phase is washed with water, then dried and evaporated to dryness. Purify by silica chromatography, eluting with cyclohexane and then with a cyclohexane/EtOAc gradient from 99/1 to 50/50 in 1 hour. We obtain 0.39 g of the expected compound. M.p.=127.4° C.

NMR: 1.27 ppm: s: 9H, 4.22 ppm: d: 2H, 5.37 ppm: bs: 2H, 6.50 ppm: s: 1H, 6.97 ppm: dd: 1H, 7.28 ppm: d: 1H, 7.36 ppm: dd: 1H, 7.46 ppm: t: 1H, 7.56 ppm: d: 1H, 7.63 ppm: d: 1H, 7.72 ppm: d: 1H.

The following tables illustrate the chemical structures and the physical properties of some compounds according to the invention. In the tables, Me, Et, iPr and tBu represent the methyl, ethyl, isopropyl and tert-butyl groups, respectively. When the compound is characterized by LC/UV/MS, operating conditions (A), (B), (C) or (D) are specified.

TABLE 2

| | |
|---|---|
| | (IA) |
| 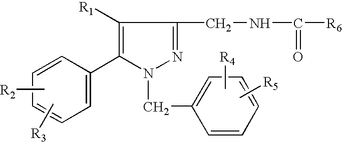 | |

| | | | | | Characterization |
|---|---|---|---|---|---|
| Com- | | | | | M.p. ° C. or |
| pounds | R₁ | R₂, R₃ | R₄, R₅ | R₆ | LC/MS |
| 1 | H | 3,4-diCl | 3-Cl, 4-Me | 4-(tBu)-phenyl | m.p. = 59° C. |
| 2 | H | 3,4-diCl | 3-Cl, 4-Me | 4-F-phenyl | m.p. = 118° C. |
| 3 | H | 3,4-diCl | 3-Cl, 4-Me | bicyclo[2.2.1] | m.p. = 107° C. |
| | | | | heptan-2-yl | |
| 4 | H | 3,4-diCl | 3-Cl, 4-Me | 4-Cl-phenyl | m.p. = 149° C. |
| 5 | H | 3,4-diCl | 3-Cl, 4-Me | 4-Me-phenyl | m.p. = 55° C. |
| | | | | | (dec.) |
| 6 | H | 3,4-diCl | 3-Cl, 4-Me | 3.5-diF-phenyl | m.p. = 130° C. |
| 7 | H | 3,4-diCl | 3-Cl, 4-Me | 2.3-diF-phenyl | m.p. = 122° C. |
| 8 | H | 3,4-diCl | 3-Cl, 4-Me | 2.5-diF-phenyl | m.p. = 140° C. |
| 9 | H | 3,4-diCl | 3-Cl, 4-Me | 2.4-diF-phenyl | m.p. = 109° C. |
| 10 | H | 3,4-diCl | 3-Cl, 4-Me | 4-MeO-phenyl | m.p. = 50° C. |
| 11 | H | 3,4-diCl | 3-Cl, 4-Me | 4-CF₃-phenyl | m.p. = 170° C. |
| 12 | H | 4-Cl | 2,4-diCl | exobicyclo[2.2.1]- | m.p. = 56° C. |
| | | | | heptan-2-yl | |
| 13 | H | 3,4-diCl | 3-Cl, 4-Me | 3,4-diF-phenyl | m.p. = 103° C. |
| 14 | H | 3,4-diCl | 3-Cl, 4-Me | 2,6-diF-phenyl | m.p. = 140° C. |
| 15 | H | 3,4-diCl | 3-Cl, 4-Me | (1,1′-biphenyl)-2-yl | m.p. = 182° C. |
| 16 | H | 3,4-diCl | 3-Cl, 4-Me | (1,1′-biphenyl)-4-yl | m.p. = 148° C. |
| 17 | H | 3,4-diCl | 3-Cl, 4-Me | 3,5-diMe-phenyl | m.p. = 95° C. |
| 18 | H | 3,4-diCl | 3-Cl, 4-Me | 4-cyclohexylphenyl | m.p. = 155° C. |
| 19 | H | 3,4-diCl | 3-Cl, 4-Me | (1-methyl)cyclohexyl | m.p. = 63° C. |
| 20 | H | 3,4-diCl | 3-Cl, 4-Me | 2,6-diMe-phenyl | m.p. = 137° C. |
| 21 | H | 3,4-diCl | 3-Cl, 4-Me | cycloheptyl | m.p. = 75° C. |
| 22 | H | 3,4-diCl | 3-Cl, 4-Me | cyclohexylmethyl | m.p. = 98° C. |
| 23 | H | 3,4-diCl | 3-Cl, 4-Me | adamant-1-yl | m.p. = 60° C. |
| 24 | H | 3,4-diCl | 3-Cl, 4-Me | cyclopentylmethyl | m.p. = 88° C. |
| 25 | H | 3,4-diCl | 3-Cl, 4-Me | tBu | m.p. = 56° C. |
| 26 | H | 3,4-diCl | 3-Cl, 4-Me | cyclohexyl | m.p. = 131° C. |
| 27 | H | 3,4-diCl | 3-Cl, 4-Me | benzhydryl | m.p. = 130° C. |
| 28 | H | 3,4-diCl | 3-Cl, 4-Me | bicyclo[2.2.1] | m.p. = 113° C. |
| | | | | octan-2-yl | |
| 29 | H | 4-Cl | 3,4-diCl | 2,6-diF-phenyl | m.p. = 165° C. |
| 30 | H | 4-Cl | 3,4-diCl | tBu | NMR |
| 31 | H | 4-Cl | 3,4-diCl | (1-methyl)cyclohexyl | m.p. = 62° C. |
| 32 | H | 3,4-diCl | 4-Cl | 2,6-diF-phenyl | m.p. = 139° C. |
| 33 | H | 3,4-diCl | 4-Cl | tBu | NMR |
| 34 | H | 3,4-diCl | 4-Cl | (1-methyl)cyclohexyl | NMR |
| 35 | H | 3,4-diCl | 3-Cl, 4-Me | 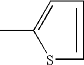 | MH⁺ = 490.40 t = 2.30 min (A) |
| 36 | H | 3,4-diCl | 3-Cl, 4-Me | cyclopropyl | MH⁺ = 448.34 |
| | | | | | t = 2.20 min |
| | | | | | (A) |
| 37 | H | 3,4-diCl | 3-Cl, 4-Me | cyclobutyl | MH⁺ = 462.41 |
| | | | | | t = 2.60 min |
| | | | | | (A) |
| 38 | H | 3,4-diCl | 3-Cl, 4-Me | 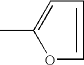 | MH⁺ = 474.08 t = 2.16 min (A) |
| 39 | H | 3,4-diCl | 4-Cl | 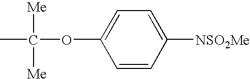 | m.p. = 80° C. |
| 40 | H | 3,4-diCl | 4-Cl | iPr | m.p. = 129° C. |
| 41 | H | 3,4-diCl | 4-Cl | —CH₂-iPr | m.p. = 119° C. |
| 42 | H | 3,4-diCl | 4-Cl | cyclopropyl | m.p. = 140° C. |
| 43 | H | 3,4-diCl | 4-Cl | 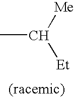 | m.p. = 120° C. |
| 44 | H | 3,4-diCl | 3-Cl | 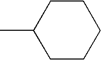 | m.p. = 143° C. |
| 45 | H | 3,4-diCl | 3-Cl | 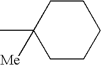 | MH⁺ = 489 t = 11.66 min (B) |
| 46 | H | 3,4-diCl | 3-Cl | tBu | MH⁺ = 449 |
| | | | | | t = 10.66 min |
| | | | | | (B) |
| 47 | H | 3,4-diCl | 3-Cl | cyclopentylmethyl | m.p. = 120° C. |
| 48 | H | 3-Cl | 3,4-diCl | 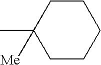 | m.p. = 93° C. |
| 49 | H | 3-Cl | 3,4-diCl | 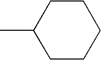 | m.p. = 125° C. |
| 50 | H | 3-Cl | 3,4-diCl | tBu | MH⁺ = 449 |
| | | | | | t = 10.63 min |
| | | | | | (B) |
| 51 | H | 3,4-diCl | 4-CF₃ | tBu | MH⁺ = 483 |
| | | | | | t = 10.87 min |
| | | | | | (B) |
| 52 | H | 3,4-diCl | 4-CF₃ | 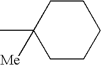 | MH⁺ = 523 t = 11.67 min (B) |
| 53 | H | 3,4-diCl | 4-CF₃ | -iPr | MH⁺ = 469 |
| | | | | | t = 10.41 min |
| | | | | | (B) |
| 54 | H | 3,4-diCl | 4-Cl | —CF₃ | m.p. = 115° C. |
| 55 | H | 4-CF₃ | 3,4-diCl | tBu | m.p. = 130° C. |
| 56 | H | 3-Cl | 3-Cl | tBu | MH⁺ = 415 |
| | | | | | t = 10.16 min |
| | | | | | (B) |
| 57 | H | 3-Cl | 3-Cl | 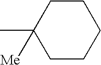 | MH⁺ = 455 t = 11.05 min (B) |
| 58 | H | 4-Cl | 4-Cl | tBu | m.p. = 97° C. |
| 59 | H | 4-Cl | 4-Cl | 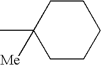 | MH⁺ = 455 t = 11.16 min (B) |
| 60 | H | 3,4-diCl | 3,4-diCl | tBu | m.p. = 56.5° C. |
| 61 | H | 3,4-diCl | 3,4-diCl | 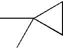 | MH⁺ = 482 t = 11.05 min (C) |
| 62 | H | 3,4-diCl | 3,4-diCl | iPr | m.p. = 113° C. |
| 63 | H | 3,4-diCl | 3,4-diCl | 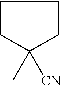 | MH⁺ = 521 t = 11.61 min (C) |
| 64 | H | 3,4-diCl | 3,4-diCl | 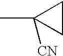 | m.p. = 125° C. |
| 65 | H | 3,4-diCl | 3,4-diCl | 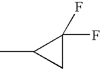 | m.p. = 140° C. |
| 66 | H | 3,4-diCl | 3-Cl, 4-OMe | tBu | m.p. = 118° C. |
| 67 | H | 3,4-diCl | 3-Cl, 4-OMe | 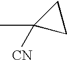 | m.p. = 143° C. |
| 68 | H | 3,4-diCl | 3-Cl, 4-OCF₃ | tBu | MH⁺ = 534 |
| | | | | | t = 11.70 min |
| | | | | | (C) |
| 69 | H | 3,4-diCl | 3-Cl, 4-OMe | 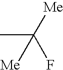 | MH⁺ = 488 t = 10.87 min (C) |
| 70 | H | 3,4-diCl | 3,4-diCl | 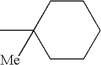 | MH⁺ = 524 t = 12.06 min (C) |
| 71 | H | 3,4-diCl | 3,4-diCl | 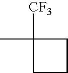 | MH⁺ = 550 t = 11.96 min (C) |
| 72 | H | 3,4-diCl | 3,4-diCl | 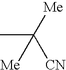 | m.p. = 89° C. |
| 73 | H | 3,4-diCl | 3,4-diCl | 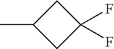 | m.p. = 143° C. |
| 74 | H | 3,4-diCl | 3,4-diCl | 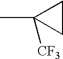 | MH⁺ = 536 t = 11.72 min (C) |
| 75 | H | 3,4-diCl | 3,4-diCl | 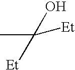 | m.p. = 82° C. |
| 76 | H | 3,4-diCl | 3,4-diCl | —CF₃ | m.p. = 100° C. |
| 77 | H | 3,4-diCl | 3-CF₃, 4-Cl | 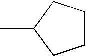 | m.p. = 89° C. |
| 78 | H | 3,4-diCl | 3-CF₃, 4-Cl | tBu | MH⁺ = 517 |
| | | | | | t = 11.28 min |
| | | | | | (C) |
| 79 | H | 3,4-diCl | 3-CF₃, 4-Cl | 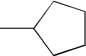 | m.p. = 92° C. |
| 80 | H | 4-OMe | 3,4-diCl | tBu | MH⁺ = 446 |
| | | | | | t = 10.07 min |
| | | | | | (C) |

NMR: Compound 30: 1.0 ppm: s: 9H, 4.1 ppm: d: 2H, 5.2 ppm: s: 2H, 6.2 ppm: s: 1H, 6.9 ppm: dd: 1H, 7.1 ppm: d: 1H, 7.2-7.6 ppm: m: 5H, 7.9 ppm: t: 1H.

NMR: Compound 33: 1.0 ppm: s: 9H, 4.2 ppm: d: 2H, 5.2 ppm: s: 2H, 6.2 ppm: s: 1H, 6.9 ppm: d: 2H, 7.2 ppm: m: 3H, 7.5 ppm: d: 1H, 7.6 ppm: d: 1H; 7.9 ppm: t: 1H.

NMR: Compound 34: 1.0-1.6 ppm: m: 11H, 1.8-2.1 ppm: d: 2H, 4.2 ppm: d: 2H, 5.3 ppm: s: 2H, 6.2 ppm: s: 1H, 7.0 ppm: d: 2H, 7.4 ppm: m: 3H, 7.6 ppm: d: 1H, 7.7 ppm: d: 1H, 8.0 ppm: t: 1H.

TABLE 3

| | |
|---|---|
| | (IB) |
| 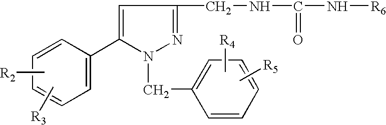 | |

| | | | | Characterization |
|---|---|---|---|---|
| Com- | | | | m.p. ° C. or |
| pounds | R₂, R₃ | R₄, R₅ | R₆ | LC/MS |
| 81 | 3,4-diCl | 3-Cl, 4-Me | tBu | m.p. = 181° C. |
| 82 | 3,4-diCl | 3-Cl, 4-Me | cyclohexyl | m.p. = 182° C. |
| 83 | 3,4-diCl | 3-Cl, 4-Me | 2,6-diF-phenyl | m.p. = 128° C. |
| | | | | (dec.) |
| 84 | 3,4-diCl | 3,4-diCl | tBu | m.p. = 150° C. |

TABLE 4

| | |
|---|---|
| | (IC) |
| 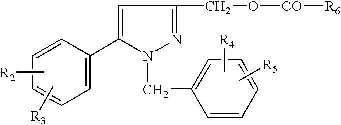 | |

| | | | | Characterization |
|---|---|---|---|---|
| Com- | | | | m.p. ° C. or |
| pounds | R₂, R₃ | R₄, R₅ | R₆ | LC/MS |
| 85 | 3,4-diCl | 3,4-diCl | 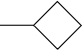 | MH⁺ = 483 t = 12.75 min (C) |
| 86 | 3,4-diCl | 3,4-diCl | 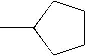 | MH⁺ = 497 t = 13.16 min (C) |

TABLE 5

| | |
|---|---|
| | (ID) |
| 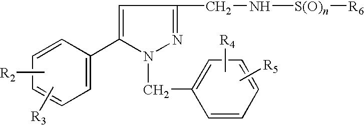 | |

| | | | | Characterization |
|---|---|---|---|---|
| Com- | | | | m.p. ° C. or |
| pounds | R₂, R₃ | R₄, R₅ | —S(O)ₙ—R₆ | LC/MS |
| 87 | 3,4-diCl | 3-Cl | —SOCF₃ | m.p. = 84° C. |
| 88 | 3,4-diCl | 3-Cl | —SO₂tBu | m.p. = 112° C. |
| 89 | 3,4-diCl | 3,4-diCl | —SOtBu | MH⁺ = 504 |
| | | | | t = 10.71 min |
| | | | | (D) |
| 90 | 3,4-diCl | 3,4-diCl | —SO₂tBu | m.p. = 127° C. |
| 91 | 3,4-diCl | 3-Cl, 4-OCF₃ | —SO₂(CH₂)₃Me | m.p. = 101° C. |
| 92 | 3,4-diCl | 3-Cl, 4-OCF₃ | —SOtBu | MH⁺ = 554 |
| | | | | t = 11.32 min |
| | | | | (C) |
| 93 | 3,4-diCl | 3-Cl, 4-OCF₃ | —SO₂tBu | MH⁺ = 570 |
| | | | | t = 11.69 min |
| | | | | (C) |
| 94 | 3,4-diCl | 3-Cl, 4-OMe | —SO₂tBu | m.p. = 59° C. |

The compounds of formula (I) possess very good affinity in vitro for the CB₂ cannabinoid receptors, whether human receptors or receptors of rodents. Affinity binding tests were carried out with membranes obtained from rodent tissues and from cell lines in which the CB₂ receptors (Munro et al., Nature 1993, 365, 61-65) were expressed, according to the experimental conditions described by M. Rinaldi-Carmona in J. Pharmacol. Exp. Therap. 1998, 287, 644-650.

More particularly, the compounds of the present invention or optional salts thereof are powerful, selective ligands of the CB₂ cannabinoid receptors, having an IC₅₀ (concentration causing 50% inhibition of the specific bond of the control) generally between 0.1 and 500 nM. They are generally between 10 and 1000 times more active on the CB₂ receptors than on the CB₁ receptors. Moreover, the antagonist nature of the compounds of formula (I) was demonstrated by the results obtained in the models of inhibition of adenylate cyclase as described in M. Rinaldi-Carmona et al., J. Pharmacol. Exp. Ther. 1996, 278, 871-878 and 1998, 284, 644-650 and M. Bouaboula et al., J. Biol. Chem., 1997, 272, 22330-22339.

The compounds according to the invention were investigated on a model of passage through the intestinal barrier constituted of Caco-2 cells. (M. C. Grès, Pharm. Res., 1998, 15(5), 726-733). This model makes it possible to define the coefficient Ptot of apparent permeability of the product for the monolayer of intestinal epithelial cells plus filter. As examples, compounds No. 60 (ex. 5) and No. 90 (ex. 7) have, in this model, a Ptot of 111.97 and 64.5 respectively (10⁻⁷ cm·s⁻¹; concentration=20 μM; apical pH=6.5 and basal pH=7.4). These values are predictive of total absorption in humans, after administration by the oral route.

Thus, according to another of these aspects, the invention relates to medicinal products for human or veterinary medicine that comprise a compound of formula (I) or a salt of addition of the latter to a pharmaceutically acceptable acid or a hydrate or a solvate. The compounds according to the invention can be used in humans or in animals (notably in mammals including but not limited to dogs, cats, horses, cattle, sheep) in the treatment or prevention of diseases involving the CB₂ cannabinoid receptors.

These medicinal products find application in therapeutics in the treatment or prevention of pathologies involving the cells of the immune system or immune disorders, for example autoimmune diseases, diseases associated with organ transplants, infectious diseases, allergic diseases, diseases of the gastrointestinal system, and diseases of inflammatory origin. More particularly we may mention the following autoimmune diseases: disseminated lupus erythematous, diseases of the connective tissue or collagenoses, Sjögren syndrome, ankylosing spondylitis, reactive arthritis, rheumatoid polyarthritis, undifferentiated spondylarthritis, Behçet disease, hemolytic autoimmune anemias, multiple sclerosis, amyotrophic lateral sclerosis (Charcot disease), psoriasis. The allergic diseases to be treated can be of the immediate hypersensitivity or asthma type, allergic rhinitis, allergic conjunctivitis or contact dermatitis. Moreover, the compounds and their optional pharmaceutically acceptable salts can be used for treating vascularities, parasitic infections, viral infections (AIDS), bacterial infections (meningitis), amyloid disease, diseases affecting the lines of the lymphohematopoietic system.

The compounds of formula (I) according to the invention can be used as medication in the treatment or prevention of pain: neuropathic pain, acute peripheral pain, chronic pain of inflammatory origin. We may mention the following inflammatory diseases: arthritis, rheumatoid arthritis, osteoarthritis, spondylitis, gout, Crohn's disease, ulcerative colitis, irritable bowel syndrome (IBS) or inflammatory bowel disease (IBD), acute pancreatitis. The compounds of formula (I) can also be used in the treatment of bone diseases and osteoporosis.

Moreover, the compounds of formula (I) according to the invention can be used as medication in the treatment or prevention of gastrointestinal disorders, diarrheal disorders, ulcers, vomiting, bladder and urinary disorders, renal disorders, renal ischemia, nephritis, disorders of endocrine origin, cardiovascular disorders, hemorrhagic shock, septic shock, chronic cirrhosis of the liver, asthma, Raynaud disease, glaucoma, fertility disorders, and as medication for anticancer chemotherapy (skin cancer, prostate cancer or cancer of cerebral origin). The compounds of formula (I) according to the invention can be used as medication in the treatment of disorders of appetite and/or of eating disorders, notably for the treatment of cachexia. The compounds of formula (I) according to the invention can be used as medication in the prevention and/or treatment of obesity and of associated cardio-metabolic diseases (hypertension, dyslipidemia, atherosclerosis), metabolic syndrome, insulin resistance (type 2 diabetes) and metabolic steatohepatopathy.

Moreover, the compounds of formula (I) can be useful as a neuroprotector, in the treatment of ischemia, head injuries and in the treatment of neurodegenerative diseases: including chorea, Huntington's chorea, Tourrette's syndrome.

The compounds of formula (I) according to the invention can be used as medication for the treatment or the prevention of migraine, stress, diseases of psychosomatic origin, panic attacks, epilepsy, movement disorders.

The compounds of formula (I) according to the invention can be used as medication in the treatment or prevention of diseases of the respiratory system such as chronic bronchitis, chronic obstructive pulmonary disease (COPD) or emphysema.

Thus, the compounds of formula (I) according to the present invention are useful in particular for the preparation of medicinal products intended for the treatment and prevention of immune disorders, pain, gastrointestinal disorders, cardiovascular or renal disorders, and/or can be used in anticancer chemotherapy.

According to another of its aspects, the present invention relates to pharmaceutical compositions comprising, as active principle, at least one compound of formula (I) according to the invention. These pharmaceutical compositions contain an effective dose of a compound according to the invention, or a pharmaceutically acceptable salt, a hydrate or solvate of said compound, as well as one or more pharmaceutically acceptable excipients.

Said excipients are selected according to the pharmaceutical form and the desired method of administration, from the usual excipients that are known by a person skilled in the art.

Apart from a compound of formula (I) or its pharmaceutically acceptable salts or hydrates or solvates, the pharmaceutical compositions according to the present invention can comprise one or more other active principles that can be used in the treatment or prevention of the pathologies mentioned above

Thus, the present invention also relates to pharmaceutical compositions comprising a compound of formula (I) according to the present invention combined with one or more active principles selected from one of the following therapeutic classes:
an antagonist and/or modulator and/or inverse agonist of the CB₁ cannabinoid receptors;
another antagonist and/or modulator and/or inverse agonist of the CB₂ cannabinoid receptors;
an antagonist of the AT₁ receptors of angiotensin II;
a converting enzyme inhibitor;
a calcium antagonist;
a diuretic;
a beta-blocker;
an antidepressant, an antipsychotic, an anxiolytic;
an anticancer agent or an antiproliferative agent;
an opioid antagonist;
   as well as:
an agent for improving memory;
an agent for use in the treatment of alcoholism or of withdrawal symptoms;
an agent for use in the treatment of osteoporosis;
an agent for use in the treatment of autoimmune diseases;
an agent for use in the treatment of organ graft rejection;
a nonsteroidal or steroidal antiinflammatory;
an antiinfectious agent;
an antiparasitic agent;
an antiviral agent;
an analgesic;
an antidiarrheal agent;
an antiasthmatic agent.
   According to another aspect of the invention, the compound of formula (I), or one of its solvates or hydrates and the other combined active principle can be administered simultaneously, separately or spread over time.

“Simultaneous use” means the administration of the compounds of the composition according to the invention contained in one and the same pharmaceutical form.

“Separate use” means the administration, at the same time, of the two compounds of the composition according to the invention, each contained in a separate pharmaceutical form.

“Use spread over time” means the successive administration, of the first compound of the composition of the invention, contained in one pharmaceutical form, then of the second compound of the composition according to the invention, contained in a separate pharmaceutical form. In this case, the time that elapses between the administration of the first compound of the composition according to the invention and the administration of the second compound of the same composition according to the invention generally does not exceed 24 hours.

In the pharmaceutical compositions of the present invention for oral, sublingual, subcutaneous, intramuscular, intravenous, topical, local, intratracheal, intranasal, transdermal or rectal administration, the active principle of formula (I) above, or its optional salt, solvate or hydrate can be administered as a unit dosage form, mixed with conventional pharmaceutical excipients, to animals and to humans for the prophylaxis or treatment of the disorders or diseases mentioned above.

The appropriate unit dosage forms comprise forms for administration by the oral route such as tablets, soft or hard capsules, powders, granules and oral solutions or suspensions, forms for sublingual, buccal, intratracheal, intraocular and intranasal administration or administration by inhalation, forms for topical, transdermal, subcutaneous, intramuscular or intravenous administration, forms for rectal administration and implants. For topical application, the compounds according to the invention can be used in creams, gels, ointments or lotions.

As an example, a unit dosage form of a compound according to the invention in the form of a tablet can comprise the following components:

| | | |
|---|---|---|
| Compound according to the invention | 50.0 | mg |
| Mannitol | 223.75 | mg |
| Croscarmellose sodium | 6.0 | mg |
| Maize starch | 15.0 | mg |
| Hydroxypropyl methylcellulose | 2.25 | mg |
| Magnesium stearate | 3.0 | mg |

For administration by the oral route, the dose of active principle administered per day can reach 0.01 to 100 mg/kg, in one or more separate doses, preferably from 0.1 to 50 mg/kg.

There may be particular cases where higher or lower dosages are appropriate; such dosages are still within the scope of the invention. According to usual practice, the appropriate dosage for each patient is determined by the doctor according to the method of administration, and the patient's weight and response.

According to another of its aspects, the present invention also relates to a method of treatment of the aforementioned pathologies which comprises the administration, to a patient, of an effective dose of a compound according to the invention, or of one of its pharmaceutically acceptable salts or hydrates or solvates.

What is claimed is:

1. A compound of the formula (I): 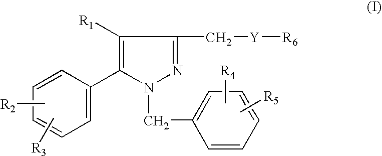
in which:
Y represents a group selected from:
i) —N(R₇)CO—,
ii) —N(R₇)CO—N(R₇)—,
iii) —OCO—, and
iv) —N(R₇)S(O)ₙ—;
R₁ represents a hydrogen atom or a (C₁-C₄)alkyl group;
R₂ and R₄ represent, each independently of one another, a hydrogen or halogen atom, or a (C₁-C₄)alkyl, (C₁-C₄)alkoxy or trifluoromethyl group;
R₃ and R₅ represent, each independently of one another, a halogen atom or a (C₁-C₄)alkyl, (C₁-C₄)alkoxy, trifluoromethyl, trifluoromethoxy, cyano or S(O)ₘAlk group;
R₆ represents a group selected from:
a (C₁-C₆)alkyl group, unsubstituted or substituted one or more times with one or more substituents selected independently from a halogen atom or a hydroxy, (C₁-C₄)alkoxy or trifluoromethoxy group;
a phenyl, unsubstituted or substituted one or more times with R₈;
a benzyl or benzhydryl;
a heterocyclic radical selected from: thienyl, furyl or pyrrolyl, said radicals being unsubstituted or substituted with a halogen atom, a (C₁-C₄)alkyl or trifluoromethyl group;
a C₃-C₁₂ nonaromatic carbocyclic radical, unsubstituted or substituted one or more times with a halogen atom or a (C₁-C₄)alkyl, (C₁-C₄)alkoxy, hydroxy or cyano group;
a (C₃-C₇)cycloalkylmethyl group, unsubstituted or substituted on the cycloalkyl with one or more (C₁-C₄)alkyl groups; and
an aryloxymethyl group, unsubstituted or substituted on the methyl with one or two alkyl groups, in which the term aryloxy represents a phenoxy group, unsubstituted or substituted one or more times with R₈;
R₇ represents a hydrogen atom or a (C₁-C₄)alkyl group;
R₈ represents a halogen atom; a (C₁-C₄)alkyl; trifluoromethyl; cyano; (C₁-C₄)alkoxy; trifluoromethoxy; phenyl; (C₃-C₇)cycloalkyl group or a group NHS(O)ₙAlk;
n represents 1 or 2;
m represents 0, 1 or 2; and
Alk represents a (C₁-C₄)alkyl;
or a salt thereof.

2. The compound as claimed in claim 1, wherein Y represents a group —N(R₇)CO—.

3. The compound as claimed in claim 1, wherein Y represents a group —N(R₇)CON(R₇)—.

4. The compound as claimed in claim 1, wherein Y represents a group —OCO—.

5. The compound as claimed in claim 1, wherein Y represents a group —N(R₇)S(O)ₙ—.

6. The compound as claimed in claim 1, wherein:
R₁ represents hydrogen;
R₂ or R₃ represents a chlorine atom at 3 or 4 position, a trifluoromethyl group at 4 position, or R₂ and R₃ are both chlorine atoms at 3 and 4 positions; and
R₄ or R₅ represents a chlorine atom at 2, 3 or 4 position, a trifluoromethyl group at 4 position, or a chlorine atom at 3 position and a methyl group at 4 position.

7. The compound as claimed in claim 2, wherein:
R₁ represents a hydrogen atom;
R₂ or R₃ represent a chlorine atom at 3 or 4 position, a trifluoromethyl group at 4 position, or R₂ and R₃ are both chlorine atoms at 3 and 4 positions;
R₄ or R₅ represent a chlorine atom at 2, 3 or 4 position, a trifluoromethyl group at 4 position, or R₄ and R₅ represent a chlorine atom at 3 position and a methyl group at 4 position; and
R₆ represents a group selected from:
a (C₁-C₆)alkyl group, unsubstituted or substituted one or more times with one or more substituents selected independently from a halogen atom or a hydroxy, (C₁-C₄)alkoxy or trifluoromethoxy group; and
a C₃-C₁₂ nonaromatic carbocyclic radical, unsubstituted or substituted one or more times with a halogen atom or a (C₁-C₄)alkyl, (C₁-C₄)alkoxy, hydroxy or cyano group.

8. A compound selected from:
N-((1-(3,4-dichlorobenzyl)-5-(3,4-dichlorophenyl)-1H-pyrazol-3-yl)methyl)-2,2-dimethylpropanamide; and
N-((1-(3,4-dichlorobenzyl)-5-(3,4-dichlorophenyl)-1H-pyrazol-3-yl)methyl)-2-methylpropane-2-sulfonamide.

9. A method of preparation of a compound of formula (I), as claimed in claim 1, comprising:
reacting a compound of formula (II): 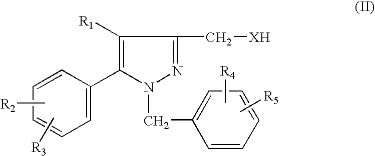
in which X represents an oxygen atom, an NH or N(R₇) group and R₁, R₂, R₃, R₄ and R₅ are as defined in claim 1:
a) either with a functional derivative of an acid of formula R₆COOH (III) in which R₆ is as defined in claim 1;
b) or with an isocyanate of formula R₆N═C═O (IV) in which R₆ is as defined in claim 1;
c) or with a halogenated derivative of formula R₆S(O)ₙHal in which Hal represents a halogen atom, and R₆ is as defined in claim 1.

10. A compound of formula (XII): 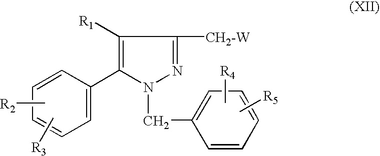
in which:
W represents a hydroxyl or amino group;
R₁ represents a hydrogen atom or a (C₁-C₄)alkyl group;
R₂ and R₄ represent, each independently of one another, a hydrogen or halogen atom, or a (C₁-C₄)alkyl, (C₁-C₄)alkoxy or trifluoromethyl group; and
R₃ and R₅ represent, each independently of one another, a halogen atom or a (C₁-C₄)alkyl, (C₁-C₄)alkoxy or trifluoromethyl group;
or a salt thereof.

11. A pharmaceutical composition comprising a compound of formula (I) as claimed in claim 1 or a pharmaceutically acceptable salt thereof and at least one pharmaceutically acceptable excipient.

12. A pharmaceutical composition comprising a compound of formula (I) as claimed in claim 2 or a pharmaceutically acceptable salt thereof and at least one pharmaceutically acceptable excipient.

13. A pharmaceutical composition comprising a compound of formula (I) as claimed in claim 3 or a pharmaceutically acceptable salt thereof and at least one pharmaceutically acceptable excipient.

14. A pharmaceutical composition comprising a compound of formula (I) as claimed in claim 4 or a pharmaceutically acceptable salt thereof and at least one pharmaceutically acceptable excipient.

15. A pharmaceutical composition comprising a compound of formula (I) as claimed in claim 5 or a pharmaceutically acceptable salt thereof and at least one pharmaceutically acceptable excipient.

16. A pharmaceutical composition comprising a compound of formula (I) as claimed in claim 6 or a pharmaceutically acceptable salt thereof and at least one pharmaceutically acceptable excipient.

17. A pharmaceutical composition comprising a compound of formula (I) as claimed in claim 7 or a pharmaceutically acceptable salt thereof and at least one pharmaceutically acceptable excipient.

18. A pharmaceutical composition comprising a compound of formula (I) as claimed in claim 8 or a pharmaceutically acceptable salt thereof and at least one pharmaceutically acceptable excipient.

19. A method for the treatment of pain, gastrointestinal disorder, cardiovascular or renal disorder in a patient comprising administering to said patient a therapeutically effective amount of a compound of formula (I) as claimed in claim 1.
